(19) United States
(12) Patent Application Publication
(10) Pub. No.: US 2013/0183399 A1
(43) Pub. Date: Jul. 18, 2013

(54) Method for Improving Physical Performance During Physical Exertion

(71) Applicants: Andrew Blow, Highcliffe (GB); Raj Jutley, Egmanton (GB)

(72) Inventors: Andrew Blow, Highcliffe (GB); Raj Jutley, Egmanton (GB)

(73) Assignee: PRECISION HYDRATION LTD., Hove (GB)

(21) Appl. No.: 13/633,002

(22) Filed: Oct. 1, 2012

Related U.S. Application Data
(60) Provisional application No. 61/627,292, filed on Oct. 11, 2011.

(51) Int. Cl. G01N 33/14; A23L 2/52

(52) U.S. Cl. CPC G01N 33/14; A23L 2/52
USPC 426/2; 426/648; 426/231; 426/66

(57) ABSTRACT

Beverages comprising sodium ion in an amount correlated to the sodium ion concentration of a subject's sweat used to prehydrate before exertion or to restore lost electrolyte and water resulting from physical exertion. Such use can avoid or substantially reduce losses in performance and, in cramp prone individuals, avoid or substantially reduce cramping brought on by physical exertion.

### CROSS REFERENCE TO RELATED APPLICATION

This application claims the benefit under 35 USC 120 of the filing date of Provisional Application Ser. No. 61/627,292, filed Oct. 11, 2011, the disclosure of which is incorporated herein by reference.

### BACKGROUND TO THE INVENTION

1. Field of the Invention

This invention relates to methods and compositions for compensating for and avoiding or minimizing the effects of loss of sodium and other electrolytes caused by physical exertion

2. Introduction

The loss of water and electrolytes, particularly sodium, during athletic competition, exercise and other forms of physical exertion can have significant consequences. Moderate losses of electrolytes can result in diminished performance and reduction in cognitive ability. High losses can cause more serious consequences up to and including death. Electrolyte losses, particularly sodium, in human sweat vary considerably from person to person, as do sweat rates. Therefore, net sodium losses over time are highly variable from individual to individual. This is especially relevant when physical work rates, and/or ambient temperatures are elevated as is often the case in sport and during manual work. Failure to substantially replace the sodium lost in sweat in such situations results in impaired cognitive function and impaired physical performance Potassium losses can increase cramping and result in cardiac irregularities. Reduction in magnesium levels is also associated with adverse effects on cardiac function and a reduction in exercise tolerance. Prolonged exercise also leads to a loss in calcium which has been associated with stress fractures, particularly in sports requiring running such as basketball and marathon running.

The need to replenish water and electrolyte losses as a result of vigorous physical activity has not gone unrecognized. Loss of water and electrolytes during exertion principally manifests itself by causing a person to feel thirsty. However, even casual athletes are usually aware that drinking water alone is not always enough to relieve thirst if a high loss of electrolytes has occurred. In such situations, drinking water, even large quantities does not “quench” the athlete's thirst because the loss of electrolytes is not restored by drinking water alone. As a result a substantial industry now exists which provides athletes and other persons who engage in rigorous physical activity with a variety of “sport drinks” comprising water and dissolved electrolytes, principally sodium. These beverages are usually flavoured to make them more palatable. Among such beverages may be mentioned beverages such as Gatorade®. Typically these beverages contain a fixed amount of sodium and the purchaser ingests a quantity which provides him/her with some level of satisfaction.

In short the consumer is presented with “one choice fits all” selection alternatives. Practically speaking, the result of this practice is that it is very unlikely that an individual will optimally replenish sodium and other electrolyte losses that occur during exercise. One reason is that individuals are unaware of their electrolyte needs and, therefore, lack the knowledge or incentive to determine the volume of electrolyte solution that should be consumed to replenish electrolyte losses. The usual result of this lack of knowledge is that individuals consume too little rehydration beverage than is necessary to replace their losses. Furthermore, and particularly in the case of persons whose sweat is very high in sodium content, the level of sodium in a particular beverage may actually be so low as to effectively require a larger volume of beverage than can practically or conveniently be consumed in any event.

To date only a limited amount of exploratory work has been done in the field of personalizing hydration and sodium replenishment regimes for athletes, manual workers or anyone else engaged in vigorous activity and/or exposed to hot environments where sweat production is increased and either physical or mental tasks need to be completed.

Herein is described a system which combines accurate analysis of sweat sodium loss with a tailored prescription of sodium and fluid replenishment products to closely match the requirements of an individual. The system decreases or eliminates the degradation in performance (mental and/or physical) that results from failure to adequately replenish fluids or sodium.

There is published evidence which indicates that sweat sodium concentrations in humans varies between individuals. For example, a study of 154 children aged between 0.5 years and 14 years 8 months², reported a mean sodium concentration of 26.8 mmol/L, with a range of 4-66 mmol/L. The range in adults is similarly broad. In a study of 175 adults, a range of sweat sodium concentrations between 12-75 mmol/L were measured (mean 36.3±14.7 mmol/L).³

In 2008, Bates and Miller evaluated 29 manual workers to quantify sodium losses during work in heat. The workers, aged 18-50 years, exercised in an environment chamber at 40% VO2 max, 50% relative humidity and 35° C. temperature. Sweat was collected from arms and legs and analysed using absorption spectrophotometry. The range of sweat sodium concentration was between 20 mmol/L to 110 mmol/L⁴. Studies with elite football (soccer) players has demonstrated similar large variation in individual sweat sodium concentrations. A study evaluating 20 elite Gaelic football players showed a sweat sodium variation of 19-52 mmol/L (mean 35 mmol/L) using sweat collected from 4 body sites⁵. Godek et al (2010) analysed sweat sodium levels in 54 American football players using sterile absorbent patches on the right forearm. A range of 15-99 mmol/L was found. This variance along with a large variation in individual player sweat volume losses translated to overall sodium losses ranging from 6.7 g/h to 642 mg/h ⁶.

As indicated above, humans’ sweat volume losses vary considerably. These variations in sweat volumes along with the variance in sweat sodium concentrations lead to a massive range of net sodium deficits. One of the more dramatic findings, reported by Godek et al (2010), demonstrated that during training sessions lasting 4.5 h, sodium losses due to sweat from individual professional footballers ranged from 2.3 to 30 g. This variation in net sodium losses has been supported by additional studies looking at sweat and sodium losses in other sports. In 2004 Maughan studied 24 Premier League football players training in warm weather with moderate humidity (24-29° C.; 46-64%). The mean sodium loss (NaCl equivalent) in the training session was 5.8±1.4 g with mean sweat losses of 2033±413 ml.⁷ The study concluded that “ . . . sweat losses of water and solute in football players in training can be substantial but vary greatly between players even with the same exercise and environmental conditions”. Even at lower temperatures the losses are significant. During a Premier League reserve game played at 6-8° C. and relative humidity 50-60%, the sodium losses in 22 players was found to be 2.4±0.8 g.⁸ A recent study on 24 ice hockey players playing for an average of 21.4 min showed that even short bursts of intense workout leads to large sodium imbalances. In this study the losses were 3.1±0.4 g (approximately 8 g NaCl).⁹ A study in 29 National Basketball Association (NBA) level players playing for 20 minutes in a cool environment (temperature 5° C., relative humidity 81%) demonstrated similar results. The players sweat sodium levels ranged from 21.3 to 58.1 mmol/L (mean 41.6±11.5 mmol/L). Sweat loss ranged from 1.0 to 4.6 L, with a mean sweat loss of 2.2±0.8 L. In the 20 minutes of play, total sodium loss ranged from 764 mg to 3.7 grams. The authors translated this to salt (NaCl) losses ranging from 1.9 to 9.5 grams during the session (mean 4.8±2.3 g).¹²

Along with variation in sweat sodium level and sweat volume losses, there is considerable variation in individual rehydration practices. Current evidence suggests that many athletes start a training session or competitive game hypohydrated.¹²⁻¹⁵ In a study evaluating pre-practice hydration status of 263 National Collegiate Athletic Association (NCAA) Division I athletes, Volpe et al (2009) showed that 53% appeared hypohydrated as evidenced by urine specific gravity. Similarly, Osterberg et al (2009) found that approximately 50% of the NBA basketball players evaluated prior to match play were found to be hypohydrated. In the Osterberg study, the mean sweat volume exceeded 2 litres in the 20 minutes average of play. The current published literature shows that most sportspeople consume significantly less liquid during activity than is required to replace sweat loss, even with adequate drinks and free access to fluids. Data collected from 26 professional soccer players training for 90 minutes in the heat showed that only 45±16% of the sweat volume loss was replaced (range 9% to 73%)¹⁶. Maughan et al (2004, 2005) replicated this wide inter-individual variation finding in cooler ambient temperatures and concluded that voluntary fluid intake is “ . . . generally insufficient to match fluid losses”. A survey of collegiate athletes has suggested that more targeted education regarding hydration is necessary to improve knowledge, attitudes, and behaviors.

With the wide variation in sodium sweat concentrations, sweat volume and inter-individual variation in drinking patterns, there is a need for individualised hydration strategies to maximise performance. Starting in a hypohydrated state with subsequent inadequate replacement of fluids while losing large sweat volumes leads to dehydration with performance impairment. This typically occurs in events lasting greater than 90 minutes in temperate climates (20-21° C.) and around 60 minutes in hot climates (31-32° C.) although it has been suggested that athletes tolerate the same level of dehydration better in temperate than hot events^{22, 23}. Over hydration with low-sodium or worse still, plain water, can lead to low serum sodium levels (exercise associated hyponatraemia—EAH). This causes water to move into the brain cells resulting in swelling. Typically the athlete experiences poor coordination, mental confusion, and muscle weakness. In extreme cases EAH can result in coma, seizures and even lead to death. To date, there are no studies investigating a direct relationship between athletes with high sweat sodium loses and EAH. However, a recent paper by Pahnke et al (2010) demonstrated that serum sodium reduction in 46 Ironman athletes competing at high temperature was directly related to sweat sodium losses. However, the effect of sodium supplementation in maintenance of serum sodium levels remains unclear. In the 2001 South African Ironman, 145 competing athletes volunteered for a study which randomised the athletes to either a placebo or a sodium supplementation (244 mg per tablet) group. The study showed no difference in finishing time, serum sodium concentration before and after the race, or weight before and during the race²⁸. The supplementation group consumed an extra 3.6 grams of sodium during the duration of the race equating to less than 300 mg sodium on average per hour. Current published evidence in competing or training athletes points to average sweat sodium concentration of around 35-40 mmol/L (805-920 mg/L) with hourly sweat sodium losses from as low as 600 mg/h to over 6000 mg/h^{3, 5, 6, 9, 12}. Therefore the average supplementation of 300 mg/h in the study group was below the published reference range and perhaps too low to have a meaningful effect on serum concentration. Also, the athletes sweat sodium loss was not measured and all groups were allowed free access to sports drinks and nutrition during the race but no record was kept of their intake. Indeed, one competitor in the placebo group developed hyponatraemia requiring medical attention.

### Role of Sodium in Athletic Performance

Despite the current published work on sweat sodium losses, variation in sweat volumes and drinking patterns, there is limited published data on the effects of sodium on athlete performance. There are however two notable studies that demonstrate a clear relationship between sodium deficits and cramping. In an observational study, Stofan et al (2005) compared 5 heat-cramp prone NCAA football players with matched players who had never cramped. Forearm sweat samples were obtained and the fluid balances were measured during a 2-day training camp. The net sodium loss for a practice session in the cramp-prone players was significantly higher than the control group (5.1±2.3 g vs. 2.2±1.7 g). The cramp-prone group also had twice the sweat sodium concentration than the control population (54.6±16.2 vs. 25.3±10.0 mmol/L). A similar study, again in football players training in the heat for 2.2 hours, showed a significant decline in plasma sodium levels in players that cramped whilst the reference (non-cramp-prone) group maintained their plasma sodium levels¹¹.

In a within-subject, placebo controlled randomised study, Snell et al (2010) evaluated the effects of fluid replenishment after moderate dehydration with either plain water, a popular sports drink (Gatorade) or an electrolyte replacement drink (Rehydrate). Only Rehydrate restored the baseline VO2 max in the test subjects. The study concluded that sodium was important for maximal exercise performance and effective recovery. Interestingly, Gatorade, which contains 110 mg sodium per serving (240 ml) compared to Rehydrate (105 mg per 240 ml serving), resulted in better performance than plain water but not to the levels seen with Rehydrate. This suggests that other factors, such as simple transportable monosaccharides along with sodium are important for performance enhancement and recovery.

A study undertaken by Kenefick et al in collaboration with the US Army in 2007 compared the effects hydration of varying tonicity on exercise performance in the heat. The study compared the effects of intra-venous (IV) 0.9% saline, IV 0.45% saline and oral 0.45% hydration fluid (containing over 1.8 g sodium per litre) in subjects performing at 50% VO2 max in an environment chamber. Ingestion of oral 0.45% fluid resulted in a significantly greater exercise time than if no fluid was consumed. Interestingly, IV administration does not offer any advantages over oral ingestion on performance and cardiovascular parameters (Kenefick 2007 and 2006).

A higher dietary sodium load does appear to confer some physiological benefits. In a review of dietary sodium and its effects on exercise in 1997, Luetkemeier states that “ . . . higher Na+ diet (also) appears to accelerate the cardiovascular and thermoregulatory adaptations that accompany heat acclimation or short term exercise training”.³² Part of the benefits may be explained by the increased hydration effects seen by including sodium in athlete drinks. Several studies have documented this finding such that the inclusion of sodium in sports drinks to increase and maintain hydration compared to water alone ‘remains the recommendation of governing bodies such as the American College of Sports Medicine.³²⁻³⁵ Moreover, sodium appears to increase palatability of sports drinks leading to increased voluntary drinking.³⁶

One of the most convincing studies on the effects of sodium supplementation on endurance is a study published in 2005 by Coles and Luetkemeier. This study evaluated 14 cyclists performing a 15 min time trial. The cyclists were randomized to a sodium-free drink or a pre-exercise 164 mmol/L sodium drink. Each cyclist undertook a 45-min session in a climate chamber at 70% maximal workload followed by a 15 min performance time trial. The cyclists who were pre-loaded with sodium showed a significant improvement of around 7.8% performance gain compared to those that ingested the sodium-free drink. These performance gains were however not replicated in a study undertaken by Loughborough University. The drinks with additional 30-40 mmol/L sodium chloride did increase hydration compared to a sodium-free drink but did not appear to result in improved performance when exercised to exhaustion.³⁸ A critical analysis of this study is that the sodium supplementation used is well below the mean levels described in other trials (30-40 mmol/L sodium).^{3, 5, 6, 9, 1} The 30-40 mmol/L NaC1 used in the study equates to only 20 mmol/L sodium making the supplementation likely to be inadequate in the 4 subjects studied especially as no sweat sodium analysis of the subjects was undertaken.

### The Role of Sodium in Cognitive Function

The effect of sodium supplementation on cognitive performance has been demonstrated in a recent study by Pahnke et al (2008). After forceful dehydration in a hot climate chamber, the study subjects were asked to complete a Stroop Color-Word test which assesses reaction times to process complex tasks. The test response times were better in subjects who maintained their serum sodium levels with sodium supplementation compared to the placebo group where serum levels dropped.

### Sweat Sodium Measurement—Current Methods

There are two methods for sweat collection and analysis described in the medical literature. One method is patch testing whereby absorbent patches are placed on body parts and once saturated, sent to a laboratory for analysis by spectrophotometry. This method appears to be the most popular one described in the medical literature when testing athletes or sportspeople.^{5, 10, 11, 12} The second method is used for diagnosis of cystic fibrosis using the Macroduct™ system manufactured by Wescor, an Elitech Company (Utah, USA).^{2, 3} This system uses a sweat inducer that delivers pilocarpine by iontophoresisis into a small area of skin to induce sweat secretion. The sweat is then collected by a Macroduct™ collecting device into a capillary tube. Once sufficient sweat is obtained the sweat is analyzed by expressing it into the Sweat-Chek™ analyzer. This measures the total electrolyte of the sweat specimen and displays the reading in equivalent NaCl molarity units (mmol/L).

The Macroduct™ system is presently the industry standard for the diagnosis of cystic fibrosis. It has been through several clinical evaluations to demonstrate efficacy. The use of the Macroduct™ system outside the diagnosis of cystic fibrosis has not been described in the scientific literature. However, the use of the Macroduct™ sweat collection pod has been documented by Bates when collecting sweat from actively exercising manual workers.⁴ In a study evaluating a control group with no cystic fibrosis, Riedi used the Macroduct™ system to collect and analyse sweat in healthy adults (no age range supplied). In the 175 control subjects tested the NaCl equivalent mean as tested by the Sweat-Chek™ analyzer was 40.9±14.1 (range 16-75 mmol/L). Sodium analysis in the same sample by flame spectrometry showed the levels in the same group to be 5 mmol/L lower (mean 36.6±14.7; range 12-75 mmol/L). On average, the sodium value was 0.89 of the NaCl value obtained by Macroduct™ system testing.³ The mean values and ranges obtained by the Macroduct™ system correlate well with the values (around 30-40 mmol/L mean; range 20-75 mmol/L sodium) found by patch testing in athletes and sportspeople.^{5, 6, 9, 10, 12}

There are commercial enterprises offering sodium sweat analysis using postal patch testing. Medion Corporation (www.medioncorp.com) is a Canadian-based company which sells patches and these absorbent patches are placed on 3 body parts and the client exercised until the patches are saturated (usually within 30-60 min). The patches are them removed and sealed in a plastic bag before mailing to the laboratory within 48 hours. There is no published work in the medical literature on this particular test kit although in the February 2010 issue of Canadian Running, an editor put the kit to test and obtained a value of 1102 mg/L sodium. He also underwent an intensive lab-based test at the Gatorade Canada's Sports Science Institute. He obtained a range of values from 2,069 mg/L of sodium from the forehead patch, 1,908 mg/L from the back, and 1,563 mg/L from the chest.⁴³ This shows a large variation (almost a doubling) of the results obtained by Medion patch testing and lab-based tests. Another facility offering sweat patch analyses is HFL Sports Science—a USA and UK-based drug surveillance laboratory. It offers as part of its service a sodium sweat analysis using a postal kit.⁴⁴ No results appear to have been published by the laboratory in the medical literature relating to this particular service. In fact, any publications describing sweat patch testing for sodium do not refer to a particular commercial service. The papers tend to describe their methodology and the spectrometer used in the study.¹²

In 2010, Schazmann described an electrochemical sensor for the real-time measurement of sweat sodium concentration. The belt collects and analyses sweat sodium levels in real-time using an ion selective electrode. Part of the trial compared sweat sodium levels in 2 CF and 4 non-CF adults.⁴⁵ The 4 controls registered a mean of sweat sodium of 26±6 mmol/L which is slightly lower than the values obtained by other groups using flame spectrometry or the Macroduct™ system. However, the sample size in this trial was small and therefore subject to bias. It did correlate well with the mean values described in the non-cramping group in the study by Stofan.¹⁰ This technology appears to be experimental with no clinical trials described in the medical literature.

A method for analyzing sweat electrolytes has been lodged as a patent application by Edmonson and Stoddard in 2008.⁴⁶ This assembly consists of a sensor head that collects sweat passively in actively sweating people. The device also has electrodes that measure the conductivity or resistance of collected sweat. No trials using this novel device appear to have published.

### SUMMARY OF THE INVENTION

The present invention provides a method for improving a subject's performance during a period of physical exertion comprising consumption by the subject of an aqueous beverage comprising dissolved sodium ion in an amount determined to optimize said performance, the sodium ion concentration in said aqueous beverage being correlated to a previously measured sweat sodium ion concentration of the subject under controlled conditions. The aqueous beverage, optionally and preferably, comprises other electrolytes lost in sweat or lost from metabolic processes occurring during exercise. Such other electrolytes include, by way of example, magnesium, potassium and calcium. The cationic electrolytes may be incorporated in the beverage in a variety of salt forms. Preferred salts are bicarbonates, carbonates, citrates, chlorides, phosphates and sulfates, including combinations thereof Some of these electrolytes are also lost in sweat during exertion and their replenishment can contribute to better performance as well. Others contribute other advantages to the beverage such as flavour improvement, carbonation, or provide useful buffering. The beverage can also contain other additives such as taste and palatability enhancers and, particularly if it is to be consumed before exertion, contain carbohydrates. Beverages consumed post exertion can also contain proteins.

The present invention also provides a method for reducing or eliminating cramping during a period of physical exertion comprising consumption by the subject of an aqueous beverage comprising dissolved sodium ion in an amount determined to optimally eliminate or reduce said cramping, the sodium ion concentration in said aqueous beverage being correlated to a previously measured sweat sodium ion concentration of the subject under controlled conditions. The aqueous beverage, optionally and preferably, comprises other electrolytes lost in sweat or which are lost from metabolic processes occurring during exercise. Such other electrolytes include, by way of example, magnesium, potassium and calcium. The cationic electrolytes may be incorporated in the beverage in a variety of salt forms. Preferred salts are bicarbonates, carbonates, citrates, chlorides, phosphates and sulfates, including combinations thereof

The invention also provides a method for determining the sodium ion concentration and volume of an aqueous beverage to be consumed by a subject during a period of physical exertion comprising measuring the sweat sodium ion concentration of the subject under controlled conditions and correlating the sweat sodium ion concentration to the volume and sodium ion concentration of the aqueous beverage that provides substantial rehydration and sodium replenishment during physical exertion. As noted above, the aqueous beverage can contain additional cationic electrolytes such as magnesium, potassium and calcium and the cationic electrolytes can be in the form of salts, for example, bicarbonates, carbonates, citrates, chlorides, phosphates, sulfates and combinations thereof.

In another aspect the invention provides compositions for rehydration and replenishment of electrolytes lost as a result of physical exertion and/or for prehydration prior to exertion, in either case to reduce or avoid the consequences of sodium ion losses that occur during exertion. The composition comprises sodium ions and, optionally, other cations such as potassium, magnesium, and calcium lost during athletic competition, exercise or other forms of physical exertion. The anionic complement of the cations are selected from anions such as, but not necessarily limited to bicarbonates, carbonates, citrates, chlorides, phosphates, sulfates. The composition is adapted to be soluble in water, for example, in the form of a powder or tabletted to be soluble in water and contain binders, solution aids and the like, or may additionally comprise water to permit consumption without further processing by the user. The amount of sodium in the composition varies depending upon the population of persons for whom it is intended. The populations of persons to which the composition is directed are determined by the sodium volume in the sweat lost by them during physical exertion determined in the manner described herein. If water is not already included in the composition, the composition is intended to be dissolved in water to provide an aqueous beverage to be consumed preferably before and during physical exertion to prevent or delay onset of the effects of electrolyte loss resulting from exertion and/or to alleviate those effects if they occur.

In another embodiment, the invention comprises a composition for the elimination or reduction of cramping during physical exertion, the composition comprising sodium ions and, optionally, other cationic ions such as potassium, magnesium, and calcium. The anionic complement of the cations are selected from anions such as, but not necessarily limited to bicarbonates, carbonates, citrates, chlorides, phosphates, sulfates. The composition is adapted to be soluble in water, for example, in the form of a powder or tabletted to be soluble in water and contain binders, solution aids and the like, or may additionally comprise water to permit consumption without further processing by the user. The amount of sodium in the composition varies depending upon the person or population of persons for whom it is intended. The person or population of persons to which the composition is directed is determined by the sodium volume in the sweat lost by the person or persons in the population during physical exertion determined in, for example, the manner described herein. If water is not already in the composition, the composition is intended to be dissolved in water to provide an aqueous beverage to be consumed preferably before and during physical exertion to prevent or delay onset of cramping or to alleviate cramping if it occurs.

The invention further comprises a method for formulating a composition for the treatment or avoidance of cramping during physical comprising combining water and a water soluble composition that comprises soluble sodium ion, the amount of sodium in said composition being correlated to a previously measured sweat sodium concentration of a subject under controlled conditions such that when the composition is dissolved in water the sodium ion concentration and water are sufficient to approximately replace the loss of water and sodium resulting from said exertion. The method for determining the sodium volume of sweat can be a method described herein. The amount of sodium in the composition varies depending upon the person or population of persons for whom it is intended. The person or population of persons to which the composition is directed are determined by the sodium volume in the sweat lost by the person or persons in a population during physical exertion determined in, for example, the manner described herein. If water is not already included in the composition, the composition is intended to be dissolved in water to provide an aqueous beverage to be consumed preferably before and during physical exertion to prevent or delay onset of cramping or to alleviate cramping if it occurs. Other cationic electrolytes can be included in the composition. Such other cationic electrolytes and suitable anionic complements are mentioned hereinabove.

The invention further comprises a method for delaying or ameliorating hyponatraemia that results from the loss of sodium ion during prolonged physical exertion. The method comprises administering to a subject an aqueous beverage comprising dissolved sodium ion in a concentration correlated to the subject's sweat sodium ion concentration determined under controlled conditions.

### BRIEF DESCRIPTION OF THE DRAWINGS

FIG. 1A is a graph showing the NaCl equivalent for athletes during physical exertion;

FIG. 1B is a graph showing the range of sweat sodium for athletes shown in FIG. 1A; and

FIG. 2 is a graph showing the sweat sodium levels of athletes during physical exertion.

### DETAILED DESCRIPTION OF THE INVENTION

In a preferred embodiment of the invention, an individual desiring to optimize his/her performance during competition or other exertion is tested under controlled conditions to determine the sweat sodium content of their sweat, usually in millimoles per litre (mmol/l), under controlled conditions. We have used the Macroduct™ and Nanoduct™ systems for testing sweat sodium concentrations in elite athletes. To date we have performed over 500 sweat tests on sporting disciplines ranging from motorsport, triathlon, endurance running and team sports such as soccer, rugby and cricket.

All tests described herein were performed on physically active people and elite athletes competing at the highest level of the sport nationally and internationally including national squads. Therefore the observations we have made apply as well to persons who engage in occupational or other activities that require substantial physical exertion and elite athletes. Sweat rate is determined, for example, by measuring net weight loss over time during a period of exercise or exposure to elevated temperature, for example an hour of exercise or temperature exposure or a combination of both. Net weight loss is determined by comparing the difference in weight before and after exertion and be adjusted for the weight of any food consumed during exercise and urine losses. Based on these measurements, we have found that electrolyte concentration in and the volume of rehydration beverage required can readily be determined for individual subjects. However, we have also found that, based on the sodium content of sweat, persons can be conveniently classified into groups which permits determination of the sodium content of a limited number of formulations which will adequately serve the great majority of subjects without the need to resort to individualized prescriptions. We have found that generally satisfactory results can be obtained by classifying individuals in one of four groupings for this purpose. These groups can be denominated as follows:

Low Salt Sweaters: 10-30 mmol/l NaCl

Low-Medium Salt Sweaters: 31-45 mmol/l NaCl

Medium-High Salt Sweaters: 46-59 mmol/l NaCl

High Salt Sweaters: 60-higher mmol/l NaCL

Those skilled in the art will appreciate that additional levels of segregation of persons into groups based on the sodium content of their sweat may provide further assurances that an individual's needs will be met. However, it is our experience that many persons engaged in physical exertion lack the opportunity to benefit from “fine tuning” their rehydration requirements because of the inability to suspend activity for the purpose of rehydrating frequently or at fixed intervals.

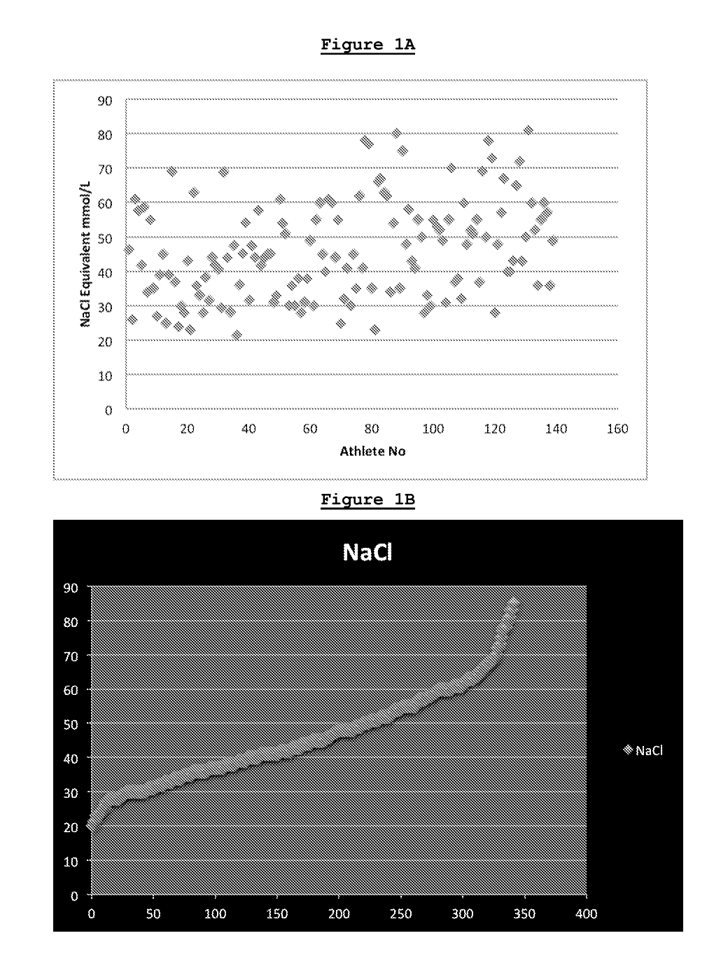

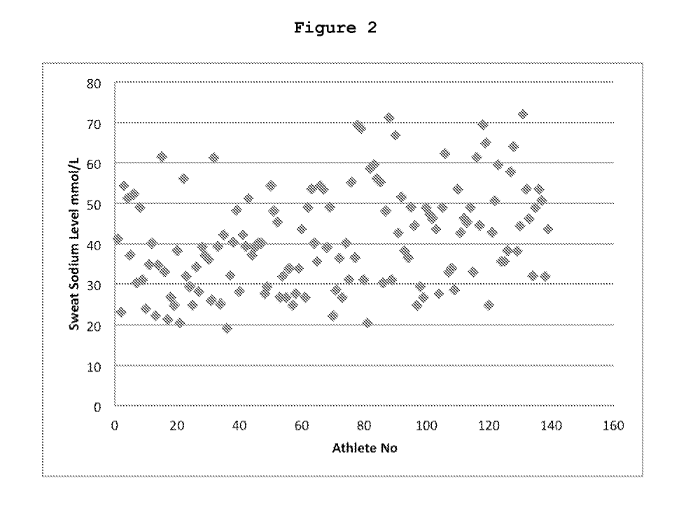

Seventy six females and 63 males were tested from our initial 139 athletes. The mean NaCl equivalent for all 139 athletes was 46.0±14 9 mmol/L (range 21-81 mmol/l). The values for the athletes are shown in FIG. 1. Using the value of 0.89 to correct NaCl to Na as seen in the paper by Riedi³, this gives a mean sweat sodium level of 41.1±12.6 mmol/L (range 19-72 mmol/l). The scatter plot is shown in FIG. 2.

From 342 tests undertaken in athletes and professional squads, an evaluation of sweat sodium levels was made between those that described as cramping ‘Often’ or ‘Always’ and those the ‘Never’ cramped. 146 athletes had no history of cramping while 71 cramped ‘Often’ or ‘Always’.

The mean sweat NaCl reading on the Macroduct system in the ‘Often’ or ‘Always’ crampers was 52.5 mmol/L and 42.5 mmol/L in the ‘Never’ group. This was a statistically significant with a p-value of <0.001 (Wilcoxon Rank Sum test) and supports our earlier findings in the previous patent (mean 56.2±15.1 vs. 41.0±11.6).

Salt Residue on Skin

An analysis was undertaken to assess if salt residue on the athlete's skin is an indicator of high sweat sodium levels. Athletes who reported a salt residue had a mean of 48.8±16.0 while those that did not had a mean of 42.9±12.9. Statistical testing did not show significance although the p-value approached significance at 0.07 (2-tailed Mann-Whitney Test).

Salt Craving

Athletes with salt craving did not have higher sweat sodium losses. Those with cravings had a mean of 47.8±15.6 while those without had means of 48.0±14.2 (p=0.8; 2-tailed Mann-Whitney Test).

Performances Fade in the Heat

Athletes describing a clear history of performance fade in the heat did not have a higher sweat sodium level compared to those with no performance fade. Mean 46.8±16.4 vs. 47.9±14.4 ((p=0.7; 2-tailed Mann-Whitney Test).

Sex Distribution

Interestingly, males (n=63) had a significantly higher sweat sodium level compared to females (n=76). Mean 51.7±14.8 vs. 41.6±12.0 (p<0.001; 2-tailed Mann-Whitney Test).

Therefore, the data by sweat testing for elite athletes along with hydration formulations for optimal rehydration treatments indicates a system can be developed for individualized performance optimization.

The System

The Precision Hydration (PH) system utilizes the following process to tailor a hydration and sodium replenishment solution for an individual:

1) Sweat analysis is conducted by obtaining a sweat sample from the individual and measuring the NaCl content, preferably using the Wescor-Ellitech Macroduct™ or Nanoduct™ system although other tests providing similar results can be used in the practice of the invention.

2) The sodium equivalent is calculated from this NaCl reading and extrapolated to give a net sodium loss per litre (milligrams, millimoles or milliequivalents per liter of sweat.

3) The individual's sweat sodium concentration is categorized into 1 of between 3 and 8 categories (Ranging very low to very high). As indicated above, preferably at least four classifications of sweat sodium concentration are used.

4) The individual's sweat rate is determined (either by estimation or measurement of weight loss during a simulated exercise bout or heat exposure).

5) Various other factors including but not necessarily limited to the amount of exercise and/or heat exposure the individual undertakes, duration of exercise sessions or working shifts, pre-existing tendencies to suffering muscle cramps are recorded.

6) From the result of the sweat analysis/categorization, sweat rate data and other factors recorded in point 5 a hydration strategy built up from a suite of rehydration formulations is recommended. The preferred formulations contain various strengths of sodium from 250 to 1500 mg/litre. Recommendations are made as to what the individual should drink for background hydration on a day to day basis, for rehydration during low/moderate exercise or heat stress situations and during levels of high exertion or high heat stress and for periods of recovery post exercise. More than one formulation will be recommended to suit the individuals' different circumstances and complete their regimen.

7) The hydration strategy can be manipulated by the individual at any time by utilizing an online portal along with a mathematical formula or manually on reviewing individual performance in the face of changing circumstances where any of the inputs can be modified in the face of changing circumstances and behaviors (for instance competing in a longer race, increasing or decreasing volume of exercise undertaken, travelling to a much hotter/cooler climate).

The compositions of the present invention contain or are intended to be dissolved in water. As noted above, the concentration of electrolytes in a particular formulation are intended to substantially restore the amount of an electrolyte, particularly sodium, that is lost during exertion and to replace the volume of water lost as sweat and by other metabolic processes such as, for example, urination. Preferably, the compositions comprise, as an aqueous solution, from at least about 200 mg of sodium per liter of water to about 2000 mg of sodium per liter of water. The compositions can be in the form of a powder or tablet but are formulated to be readily soluble in an amount of water that results in concentrations of sodium within the aforementioned range. As noted, the compositions may contain other cationic electrolytes, particularly potassium, magnesium (as Mg⁺² and calcium (as Ca⁺²) in addition to sodium. Preferably, the ranges of the amounts of these cations per liter of water are:
Potassium 100 to 700 mg
Calcium 20 to 250 mg
Magnesium 5 to 125 mg
   Presently preferred drink formulations (Per 1000m1) include the following:

### Formulation (H2ProHydrate 250)

Sodium 250 mg
Potassium 125 mg
Calcium 24 mg
Magnesium 12 mg

### Formulation 1 (H2ProHydrate 500)

Sodium 500 mg
Potassium 250 mg
Calcium 50 mg
Magnesium 25 mg
   Salts used:
Sodium Hydrogencarbonate
Potassium Hydrogencarbonate
Calcium Carbonate heavy
Magnesium Carbonate heavy

### Formulation 2 (H2ProHydrate 1000)

Sodium 1000 mg
Potassium 400 mg
Calcium 100 mg
Magnesium 50 mg
   Salts used
Sodium Hydrogencarbonate
Potassium Hydrogencarbonate
Calcium Carbonate heavy
Trimagnesium citrate anhydrous

### Formulation 3 (H2ProHydrate 1500)

Sodium 1500 mg
Potassium 600 mg
Calcium 200 mg
Magnesium 100 mg
   Salts used:
Sodium chloride
Sodium carbonate anhydrous
Potassium Hydrogencarbonate
Calcium Carbonate heavy
Magnesium Carbonate heavy

### Athlete Testing

Customised hydration recommendations based upon sweat sodium analysis values and the formulations listed above resulted in better performance and reduced cramping. To date we have tested over 500 athletes at the highest level in their sport and physically active persons. The sporting disciplines and level of competition and occupations include:
Professional football and rugby teams
National senior and U19 women's football squads
National men's and women's rugby squad
Motorsport drivers including those at Formula 1 and World Rally Championship level
International level cricket players
Ironman level tri-athletes
Elite class cyclists
Elite level ultra-endurance athletes
Fire-fighters
Soldiers
   The recommendations made were based on the method described below.

Initially athletes/players/persons are classified solely by sweat sodium analysis as Low (L), Low/Medium (LM), Medium/High (MH) or High (H) using the mmol/L values described earlier. Each classification has its own recommendation for drinks to use as background hydration, training hydration and race/competition hydration.

If however any of the following conditions apply then an individual athlete (or group of athletes) will have their strategy up graded to the next strategy (to account for increased sodium losses or counter symptoms of low sodium such as cramping):
1) Cramper? If Yes (answers OFTEN or ALWAYS to cramping question in questionnaire) then automatically up-rate to next strategy, unless already in H.
2) Training volume >14 hr per week. If training volume exceeds 14 hours per week then automatically up-rate to the next strategy.
3) Expected ambient conditions above 27 degrees C. If Yes then automatically up-rate to next strategy (works cumulatively with other factors if applicable—e.g. a cramper in 27+ degrees C. moves up 2 classifications as would over an athlete training >14 hrs per week training in 27+ degrees C.)

### Strategy L

Low Salt Sweater (10-30 mmol/L NaCl)

| | |
|---|---|
| Background hydration (training) | Water or H2ProHydrate 250 |
| Training Hydration | H2ProHydrate 500 |
| Race hydration | H2ProHydrate 1000 |

### Strategy LM

Low-Medium Salt Sweater (31-45 mmol/L)

| | |
|---|---|
| Background hydration (training) | H2ProHydrate 250 or |
| | H2ProHydrate 500 |
| Training Hydration | H2ProHydrate 1000 |
| Race Hydration | H2ProHydrate 1000 |

### Strategy MH

Medium-High Salt Sweater (46-59 mmol/L)

| | |
|---|---|
| Background hydration (training) | H2ProHydrate 250 or |
| | H2ProHydrate 500 |
| Training Hydration | H2ProHydrate 1000 |
| Race Hydration | H2ProHydrate 1500 |

### Strategy H

High Salt Sweater (60 mmol/L Plus)

| | |
|---|---|
| Background hydration (training) | H2ProHydrate 250 or |
| | H2ProHydrate 500 |
| Training Hydration | H2ProHydrate 1500 |
| Race Hydration | H2ProHydrate 1500 |

On non-training days background hydration recommendation is usually water or H2ProHydrate 250. The only exception being when an athlete is in a period of ‘preloading’ (described below).

### Preloading Recommendations:

Sodium preloading is recommended in certain conditions such as long duration (more than 2 hours) high intensity exercise with elevated ambient temperatures where anticipated losses are likely to be greater. In this scenario it is recommended to use the same formulation as their training hydration formulation for background hydration in the 48 hours prior to the activity. For example an athlete using the M/H strategy would replace H2ProHydrate 250 or H2ProHydrate 500 as their background hydration drink with H2ProHydrate 1000 for 48 hours prior to competition only.

### Volume Recommendations:

Athletes sweat rates vary considerably from person to person and for the same person in different ambient conditions and at different work rates. Replacement of sweat losses can be governed by subjectively using thirst as an indicator, or objectively using weight loss, urine osmolality or specific gravity, urine colour (Urine Armstrong Chart) amongst other established methods. Furthermore, the volumes to be replaced vary from sport to sport. For example, in a short duration high intensity sport, the athlete may choose not to replace 100% of losses. We make broad recommendations on volume replacement using established principles:

1) Drink to thirst the prescribed H2Pro fluids depending on whether they are background fluids, training fluids or race/competition fluids.
2) Aim to maintain pale urine output (Grade 1-3 on Armstrong Urine Chart).
3) Aim to replace between 70 and 100% of fluid losses measured by bodyweight during a training session or race/game.
   What we are most concerned with doing is closely matching the composition of the fluids they are consuming to what is being lost in order to restore both sodium and fluid volumes efficiently.
1) Sweat testing along with drink Formulations results in better athletic performance.
   a) Small group study. Three “high salt sweaters” (>70 mmol/L) were evaluated in a randomized cross-over study. The athletes were allowed 72 hours of pre-test hydration along with ad libitum fluid intake using either H2ProHydrate 1500 versus a commercial drink (Elite Water containing 125 mg/L of sodium). The athletes were subjected to a 30 degree Celsius heat chamber for 60-90 minutes and were then placed on a 90 minute run with pace fully controlled by the athlete. Following the consumption of ad libitum H2ProHydrate 1500 containing 1500 mg sodium per litre during the chamber exposure, there was a 5%-8% increase in the distance run by the athletes, which corresponds to approximately 1 additional mile distance in 90 minutes.
   b) Individual performance gain—lab study. A ‘high salt sweater’ ex-Ironman and international elite athlete was asked to drink a regular low sodium sports drink (Gatorade) for 3 days while maintaining a regular diet and lifestyle. The athlete was then exposed to a performance treadmill test in a heat chamber at hot and humid simulated conditions and exercised to exhaustion. The test was repeated 2 weeks later but this time the athlete prescribed ad libitum Formulation Drink with sodium levels appropriate to his sweat test (H2ProHydrate 1500 containing 1500 mg sodium per litre) which was consumed during the 2 week interval while maintaining the same diet and lifestyle as previous to the H2ProHydrate 1500 consumption. The athlete achieved a greater distance under the same heat, humidity and exercise conditions as above which was linearly extrapolated to an extra 1.3 miles at 90 minutes.
   c) Team sport performance gain (evidence provided by squad doctor). A mid-20's female playing for an international rugby squad previously fatigued at 40-60 minutes and had to be called off the field for performance fade. At this stage she would complain of cramp while also ‘running out of steam’. She was subjected to several medical tests with negative results. During squad sweat testing she was found to be a high salt sweater and prescribed a hydration strategy appropriate to her composition and volume losses. The strategy consisted of a high sodium drink equivalent to H2ProHydrate 1000 (1000 mg sodium/L). Since then she has played full games and remains a key try scorer. Her performance is more stable and predictable.
2) Sweat testing along with drink Formulations results in cramping cessation.

Individual case study.
A 36 year old businessman who is a keen endurance runner and has participated in the sport for several decades, during which he trains between 10 km to full marathons and trains 4-5 times per week (averaging 50 miles). In 2008, at 19 miles during a marathon he had cramps in his thighs and calves. In 2009 in two races these symptoms recurred at 21 miles and 23 miles distance and affected his hamstrings and latterly his left arm. In 2010, the cramps started at 18 miles and in this marathon he completed the distance but suffered headaches. His fluid regime during the races was water supplemented with salt tablets to approximately 200 mg sodium per hour.
During his running assessment he lost 2.3 kg in weight translating to 2.3 L of sweat per hour making him a high volume sweater. Sweat sodium values were measured and were 59 mmol/L making him a high salt sweater. The runner was put onto a regime which involved electrolyte consumptions using H2ProHydrate 1500 above and this strategy resulted in the runner being able to compete and complete marathons and ultra-marathons without suffering cramps. His regime was as follows:
1. Pre-race (36 hours pre-race) hydration with high electrolyte fluid containing 720 mg/L. and maintain pale urine (Armstrong Grade 1, 2 or 3).
2. Race hydration with 1000 mg/hr sodium and aim to consume at least 75% of weight lost per hour. For example, if 1.0 kg is lost per hour then replace with at least 750 mL of water.

Group analysis.
From 342 tests undertaken in athletes and professional squads, an evaluation of sweat sodium levels was made between those that described as cramping ‘Often’ or ‘Always’ and those the ‘Never’ cramped. 146 athletes had no history of cramping while 71 cramped ‘Often’ or ‘Always’.
The mean sweat NaCl reading on the Macroduct system in the ‘Often’ or ‘Always’ crampers was 52.5 mmol/L and 42.5 mmol/L in the ‘Never’ group. This was a statistically significant with a p-value of <0.001 (Wilcoxon Rank Sum test) and supports our earlier findings in the previous patent (mean 56.2±15.1 vs. 41.0±11.6).
Having looked at cramp-prone and non-crampers, we have found that the cramp-prone athletes have significantly higher sweat sodium levels compared to non-crampers. Also, net sodium losses taking into account their sweat volume loss are higher. Following the sweat analysis, the athletes were given drink formulations appropriate for their respective sweat sodium levels. To date, all “cramping” athletes (a 100% response) given the prescription of sports drink Formulation with the appropriate levels of sodium to the cramp-prone population have reported a complete cessation or dramatic reduction in their cramping symptoms. The hydration regime given to the players was to replace between 75% to 100% of their sodium losses depending on the duration and intensity of the sport/race. Those that have failed to continue their prescribed drinks have experienced rebound cramping.
3) Randomized multi-athlete trial comparing hydration with H2ProHydrate

Formulations with water.
Six male elite athletes were randomized into two cohorts in order to test the effect of electrolyte replenishment in accordance with the present invention on physical and cognitive performance during and after controlled physical exertion. The members of one of the cohorts were randomized to a hydration regimen in which they hydrated using Evian bottled water while the members of the other cohort hydrated using one of the H2ProHydrate formulations described above. The particular H2Pro formulation used by an athlete was selected based on his sweat classification determined as described above. The hydration regimen was initiated 72 hours before controlled physical exertion was initiated and maintained during the exertion period. The athletes consumed their hydration beverage ad libitum.

Just prior to beginning the controlled physical exertion the athletes were subjected to the following tests: (i) urine specific gravity; (ii) weight; (iii) serum biochemistry to determine lactate, sodium and creatinine levels; (iv) Batak Wall scoring; (v) determination of Wayne Saccatic Fixator Score; and (vi) Stroop Tests 1 & 2. The results of these tests were used as a baseline for the measurement of the effects on the athlete of the controlled physical exertion.

Following performance of the baseline tests, each athlete was placed in a heat chamber where, using a stationary bicycle, he exercised at 70% VO₂ max at 28° C. for one hour during and after which the foregoing identified tests were repeated. After 10 minutes, the athlete returned to the chamber and, using the stationary bicycle, performed a blinded (to power output) performance time trial of 15 minutes at maximum effort, following which the aforementioned tests were repeated again.

Two weeks after the initial chamber testing, the two cohorts switched hydration regimens and, again following a 72 hour period, the same baseline testing followed by controlled exertion and post exertion testing was repeated.

The sodium sweat category of the six athletes and H2Pro hydration beverage used by the athletes were as follows:

| Athlete No. | Sweat Sodium Category | Hydration Drink |
|---|---|---|
| 1 | High | H2Pro1500 |
| 2 | High | H2Pro1500 |
| 3 | High | H2Pro1500 |
| 4 | Medium | H2Pro1000 |
| 5 | Low | H2Pro500 |
| 6 | Low | H2Pro500 |

The following results were observed for the six athletes based on the results of the tests:

After 1 hour at 70% VO₂ max at 28° C.:
(a) An average of 4.4% increase in power output (watts) was observed by athletes when hydrating with an H2Pro formulation than when hydrating with water. High salt sweaters observed an even greater 8.8% power increase when using an H2ProHydrate 1500 formulation.
(b) Perceived exertion was lower (Borg Scale) for athletes on H2ProHydrate formulations (16.03) compared to perceived exertion on water (16.27). Again the results for high salt sweaters was even better on H2ProHydrate 1500 (15.5) than on water (16.4). Accordingly, hydrating with H2ProHydrate permits the athletes to improve sustained power output without greater physical discomfort.
(c) Measured mean heart rate for athletes on H2ProHydrate was 162/min, a 4.3% reduction over mean heart rate of 169/min on water; the measured mean heart rate for high salt sweaters on H2ProHydrate 1500 was 168/min compared to 158/min on water, a 6.3% reduction. Thus hydration with H2ProHydrate permitted greater sustained power output with less physiological stress.
(d) The mean core body temperature of the athletes on H2ProHydrate was 38.4° C. compared to 38.6° C., demonstrating that sustained power output using customized hydration provided by H2ProHydrate formulations results in less physiological stress.
(e) Mean lactate levels were very nearly the same (4.8 mmol/L) when the athletes were on H2ProHydrate formulations compared to measured levels when on water (4.7 mmol/L) when hydrating with water, demonstrating that higher power output can be obtained on H2ProHydrate with the same physiological stress seen when water is used for hydrating.
(f) Serum sodium levels for athletes on H2ProHydrate remained above a baseline of 141 mg/L whereas serum sodium levels for athletes on water were at baseline and after exercise under 139 mg/L. This demonstrates that use of H2ProHydrate formulations to hydrate reduces the risk of hyponatraemia.
(g) Serum creatinine levels (baseline 100) for athletes using H2ProHydrate formulations remained below 120 whereas for athletes on water the levels rose to a mean of 130. This demonstrates that sustained exercise on H2ProHydrate formulations results in less muscle breakdown than occurs with athletes on water alone.
(h) The median specific gravity of athletes urine (baseline 1.005) was lower after exercise for athletes on H2ProHydrate (1.010) than for athletes on water (1.013)indicating better hydration ofn H2ProHydrate.
   After 15 min. time trial:
(a) An average of 7.3% increase in power output (watts) was observed by athletes when hydrating with an H2ProHydrate formulation than when hydrating with water. High salt sweaters observed a significantly greater 19.8% power increase when using an H2ProHydrate 1500 formulation.
(b) Mean Batak performance for athletes on H2ProHydrate increased from the baseline for athletes on H2Pro formulations by about 5.3% whereas Batak performance of athletes on water decreased by 2.9% from the baseline for those athletes. The largest increase in Batak performance (about 7.3%) was seen for high salt sweaters. These results indicate that athletes on customized electrolyte replacement formulations increase their reaction times, particularly high salt sweaters.
(c) The athletes also were tested using Stroop 1 and 2 test regimens. Essentially no difference in performance was seen overall for athletes on water compared to athletes on H2Pro formulations. However, high salt sweaters showed an increase of about 9.5% in reaction time when on an H2ProHydrate formulation.
(d) A test for hand/eye coordination was also performed on the cohorts of athletes. Hydration with both water and customized electrolyte formulations resulted in some improvement for both dominant and non-dominant hand/eye coordination. The principal beneficial effect for users of H2Pro formulations e was seen in high salt sweaters and especially for non-dominant hand/eye coordination.

### REFERENCES

1. van der Merwe D E, Ubbink J B, De1port R, Becker P, Dhatt G S, Vermaak W J. Biological variation in sweat sodium chloride conductivity. Ann Clin Biochem. 2002; 39:39-43
2. M. Heeley, D. Woolf, A. Heeley. Indirect measurements of sweat electrolyte concentration in the laboratory diagnosis of cystic fibrosis. Arch Dis Child. 2000; 82: 420-424
3. Riedi C A, Zavadniak A F, Silva D C, Franco A, Filho N A. Comparison of conductivity with sodium determination in the same sweat sample. J Pediatr (Rio J). 2000; 76:443-6
4. Bates P B, Miller V S. Sweat rate and sodium loss during work in the heat. J Occup Med Toxicol. 2008; 29;3:4
5. Newell M, Newell J, Grant S. Fluid and electrolyte balance in elite Gaelic football players. Ir Med J. 2008; 101:236-9
6. Godek S F, Peduzzi C, Burkholder R, Condon S, Dorshimer G, Bartolozzi A R. Sweat rates, sweat sodium concentrations, and sodium losses in 3 groups of professional football players. J Athl Train. 2010; 45:364-71
7. Maughan R J, Merson S J, Broad N P, Shirreffs S M. Fluid and electrolyte intake and loss in elite soccer players during training. Int J Sport Nutr Exerc Metab. 2004; 14:333-46
8. Maughan R J, Watson P, Evans G H, Broad N, Shirreffs S M.Water balance and salt losses in competitive football. Int J Sport Nutr Exerc Metab. 2007; 17:583-94
9. Logan-Sprenger H M, Palmer M S, Spriet L L. Estimated fluid and sodium balance and drink preferences in elite male junior players during an ice hockey game. Appl Physiol Nutr Metab. 2011;36:145-52
10. Stofan J R, Zachwieja J J, Horswill C A, Murray R, Anderson S A, Eichner E R. Sweat and sodium losses in NCAA football players: a precursor to heat cramps? Int J Sport Nutr Exerc Metab. 2005; 15:641-52
11. Horswill C A, Stofan J R, Lacambra M, Toriscelli T A, Eichner E R, Murray R. Sodium balance during U. S. football training in the heat: cramp-prone vs. reference players. Int J Sports Med. 2009; 30:789-94
12. Osterberg K L, Horswill C A, Baker L B. Pregame urine specific gravity and fluid intake by National Basketball Association players during competition. J Athl Train. 2009; 44:53-7
13. Volpe S L, Poule K A, Bland E G. Estimation of prepractice hydration status of National Collegiate Athletic Association Division I athletes. J Athl Train. 2009; 44:624-9
14. Bergeron M F, Waller J L, Marinik E L. Voluntary fluid intake and core temperature responses in adolescent tennis players: sports beverages versus water. Br J Sports Med. 2006; 40:406-410
15. Stover E A, Zachwieja J J, Stofan J R, Murray R, Horswill C A. Consistently high urine specific gravity in adolescent American football players and the impact of an acute drinking strategy. Int J Sports Med. 2006; 27:330-335
16. Shirreffs S M, Aragon-Vargas L F, Chamorro M, Maughan R J, Serratosa L, Zachwieja J J. The sweating response of elite professional soccer players to training in the heat. Int J Sports Med. 2005; 26:90-5
17. Maughan R J, Shirreffs S M, Merson S J, Horswill C A. Fluid and electrolyte balance in elite male football (soccer) players training in a cool environment. J Sports Sci. 2005; 23:73-9
18. Nichols P E, Jonnalagadda S S, Rosenbloom C A, Trinkaus M Knowledge, attitudes, and behaviors regarding hydration and fluid replacement of collegiate athletes. Int J Sport Nutr Exerc Metab. 2005; 15:515-27
19. Maughan R J, Shirreffs S M. Dehydration and rehydration in competative sport. Scand J Med Sci Sports. 2010; 20 Suppl 3:40-7
20. Maughan R J, Shirreffs S M. Development of hydration strategies to optimize performance for athletes in high-intensity sports and in sports with repeated intense efforts. Scand J Med Sci Sports. 2010; 20 Suppl 2:59-69
21. Casa D J, Armstrong L E, Hillman S K, Montain S J, Reiff R V, Rich B S, Roberts W O, Stone J A. National Athletic Trainers' Association Position Statement: fluid replacement for athletes. J Athl Train. 2000; 35:212-24
22. Below P, Mora-Rodriguez R, Gonzalez-Alonso J, Coyle E. Fluid and carbohydrate ingestion independently improve performance during 1 h of intense exercise. Medicine and Science in Sports and Exercise, 1995, 27:200-210
23. Cheuvront S, Carter R, Sawka M N. Fluid balance and endurance exercise performance. Current Sports Medicine Reports, 2003, 2: 202-208
24. Hew-Butler T, Almond C, Ayus J C, et al. Consensus statement of the 1st International Exercise-Associated Hyponatraemia Consensus Development Conference, Cape Town, South Africa 2005. Clin J Sport Med 2005; 15:208-13
25. Goudie A M, Tunstall-Pedoe D S, Kerins M. Altered mental status after a marathon. N Engl J Med 2005; 352:1613-4
26. Speedy D B, Noakes T D, Schneider C. Exercise-associated hyponatraemia: a review. Emerg Med (Fremantle) 2001; 13:17-27
27. Pahnke M D, Trinity J D, Zachwieja J J, Stofan J R, Hiller W D, Coyle E F. Serum sodium concentration changes are related to fluid balance and sweat sodium loss. Med Sci Sports Exerc. 2010 September; 42(9):1669-74.
28. T D Hew-Butler, K Sharwood, M Collins, D Speedy, T Noakes. Sodium supplementation is not required to maintain serum sodium concentrations during an Ironman triathlon. Br J Sports Med 2006; 40:255-259
29. Snell P G, Ward R, Kandaswami C, Stohs S J. Comparative effects of selected non-caffeinated rehydration sports drinks on short-term performance following moderate dehydration. J Int Soc Sports Nutr. 2010; 7:28
30. Kenefick R W, Maresh C M, Armstrong L E, Riebe D, Echegaray M E, Castellani J W. Rehydration with fluid of varying tonicities: effects on fluid regulatory hormones and exercise performance in the heat. J Appl Physiol. 2007; 102:1899-905
31. Kenefick R W, O'Moore K M, Mahood N V, Castellani J W. Rapid I V versus oral rehydration: responses to subsequent exercise heat stress. Med Sci Sports Exerc. 2006; 38:2125-31
32. Luetkemeier M J, Coles M G, Askew E W. Dietary sodium and plasma volume levels with exercise. Sports Med. 1997; 23:279-86
33. Shirreffs S M, Aragon-Vargas L F, Keil M, Love T D, Phillips S. Rehydration after exercise in the heat: a comparison of 4 commonly used drinks. Int J Sport Nutr Exerc Metab. 2007; 17:244-58
34. R J Maughan, J B Leiper, S M Shirreffs Factors influencing the restoration of fluid and electrolyte balance after exercise in the heat. Br J Sports Med. 1997; 31:175-82
35. Shirreffs S M, Taylor A J, Leiper J B, Maughan R J. Post-exercise rehydration in man: effects of volume consumed and drink sodium content. Med Sci Sports Exerc. 1996; 28:1260-71
36. Passe D H, Stofan J R, Rowe C L, Horswill C A, Murray R. Exercise condition affects hedonic responses to sodium in a sport drink. Appetite. 2009; 52:561-7
37. Coles M G, Luetkemeier M J. Sodium-facilitated hypervolemia, endurance performance, and thermoregulation. Int J Sports Med. 2005; 26:182-7
38. Merson S J, Maughan R J, Shirreffs S M. Rehydration with drinks differing in sodium concentration and recovery from moderate exercise-induced hypohydration in man. Eur J Appl Physiol. 2008; 103:585-94
39. Pahnke M D, J D Trinity, J R Trombold, E F Coyle (2008). Sodium supplementation maintains serum sodium concentration and improves cognitive function in endurance athletes during exercise. Medicine and Science in Sport and Exercise. Vol 40; 5 supplement to May 2008, S86
40. Mattar A C, Gomes E N, Adde F V, Leone C, Rodrigues J C. J Pediatr (Rio J). Comparison between classic Gibson and Cooke technique and sweat conductivity test in patients with and without cystic fibrosis. 2010; 86:109-14
41. Hammond K B, Turcios N L, Gibson L E. Comparison between classic Gibson and Cooke technique and sweat conductivity test in patients with and without cystic fibrosis. J Pediatr (Rio J). 2010; 86:109-14
42. http ://www.medioncorp.com/sweat-analysis-p136275
43. http ://runningmagazine.ca/2010/02/sections/health-nutrition/body-work/lab-rat-running-theories-put-to-the-test-sweating-by-the-numbers
44. http://www.lgc.co.uk/divisions/hfl/hfl_sport_science/athletes_and_nutrition/athlete_fitness_screening.aspx
45. Schazmann, B, Morris D, Slater C, Beirne S, Fay C, Reuveny R, Moyna, Diamond D. A wearable electrochemical sensor for the real-time measurement of sweat sodium concentration. Analytical Methods. 2010; 2:342-348
46. http://www.patentstorm.us/patents/7383072/description.html

### GLOSSARY

VO2 max also maximal oxygen consumption, maximal oxygen uptake, peak oxygen uptake or aerobic capacity.

We claim:

1. A method for improving a subject's physical performance during a period of physical exertion comprising consumption by the subject of an aqueous beverage containing dissolved sodium ion in an amount determined to optimize said performance, the sodium ion concentration of said aqueous beverage being correlated to a previously measured sweat sodium ion concentration of the subject under controlled conditions.

2. A method according to claim 1 wherein the beverage further contains an ion selected from the group consisting of potassium, magnesium and calcium ions.

3. A method according to claim 2 wherein the ion comprises a salt, the anion of which is selected from the group consisting of bicarbonate, carbonate, citrate, chloride, phosphate and sulphate anions.

4. A method for determining the sodium ion concentration of an aqueous beverage to be consumed by a subject during a period of physical exertion comprising measuring the sweat volume and the sweat sodium ion concentration of the subject under controlled conditions and correlating the sweat sodium ion concentration to the sodium ion concentration of the aqueous beverage that provides substantial rehydration and sodium replenishment during physical exertion.

5. A composition for the treatment of cramping occurring during physical exertion comprising an aqueous solution of sodium ion, the sodium ion concentration of said aqueous beverage being correlated to the sweat sodium ion concentration of a subject under controlled conditions.

6. A water soluble composition for the treatment of cramping resulting from physical exertion, which composition is adapted to be dissolved in water prior to said treatment and comprises soluble sodium ion, the amount of sodium in said composition being correlated to a previously measured sweat sodium concentration of a subject under controlled conditions such that when the composition is dissolved in water the sodium ion concentration and water are sufficient to approximately replace the loss of water and sodium resulting from said exertion.

7. A method for formulating a composition for the treatment of a subject prone to cramping during physical exertion comprising combining water and a water soluble composition that comprises soluble sodium ion, the amount of sodium in said composition being correlated to a previously measured sweat sodium concentration of a subject under controlled conditions such that when the composition is dissolved in water the sodium ion concentration and water are sufficient to approximately replace the loss of water and sodium resulting from said exertion.

8. A method for avoiding or ameliorating hyponatraemia in a subject resulting from sodium loss during prolonged physical exertion comprising administering to the subject an aqueous beverage comprising dissolved sodium in a concentration correlated to the subject's sweat sodium ion concentration determined under controlled conditions.
